(19) United States
(12) Patent Application Publication
(10) Pub. No.: US 2023/0233830 A1
(43) Pub. Date: Jul. 27, 2023

(54) CATHETER SYSTEM HAVING A LOCKING CAP

(71) Applicant: Becton, Dickinson and Company, Franklin Lakes, NJ (US)

(72) Inventors: Karthik MR, Bangalore (IN); Jeevan Deshpande, Bengaluru (IN); Balaji Kannan, Chennai (IN); Sakthivel Karthikeyan, Coimbatore (IN)

(21) Appl. No.: 18/076,218

(22) Filed: Dec. 6, 2022

Related U.S. Application Data
(60) Provisional application No. 63/302,925, filed on Jan. 25, 2022.

(51) Int. Cl. A61M 39/20; A61M 39/10

(52) U.S. Cl. CPC A61M 39/20; A61M 39/10; A61M 2039/1077; A61M 2039/205

(57) ABSTRACT

A catheter system may include a cap, which may include a top. The cap may include a male luer extending distally from the top and inserted into a proximal end of a female luer adapter of the catheter system. The cap may include a first arm and a second arm extending distally from the top. The first arm may include a first slot, and the second arm may include a second slot. The first protrusion may be disposed within the first slot and the second protrusion may be disposed within the second slot. The cap may include a collar extending distally from the top. An inner surface of the collar may include a first undercut and a second undercut. The first protrusion may be disposed within the first undercut and the second protrusion may be disposed within the second undercut.

### RELATED APPLICATIONS

This application claims the benefit of U.S. Provisional Patent Application No. 63/302,925, filed Jan. 25, 2022, and entitled CATHETER SYSTEM HAVING A LOCKING CAP, which is incorporated herein in its entirety.

### BACKGROUND

A common type of catheter assembly includes a peripheral intravenous catheter (“PIVC”) that is over-the-needle. As its name implies, the PIVC that is over-the-needle may be mounted over an introducer needle having a sharp distal tip. The catheter assembly may include a catheter adapter, the PIVC extending distally from the catheter adapter, and the introducer needle extending through the PIVC. The PIVC and the introducer needle may be assembled such that the distal tip of the introducer needle extends beyond the distal tip of the PIVC with the bevel of the needle facing up away from skin of the patient immediately prior to insertion into the skin. The PIVC and the introducer needle are generally inserted at a shallow angle through the skin into vasculature of the patient.

In order to verify proper placement of the introducer needle and/or the PIVC in the blood vessel, a clinician may confirm that there is flashback of blood in a flashback chamber of the catheter assembly. In some instances, blood may travel into the introducer needle and then out of a flashback notch in the introducer needle to reach the flashback chamber, where the blood is visible to the clinician. Once placement of the introducer needle has been confirmed by observation of the blood, the clinician may remove the introducer needle, leaving the PIVC in place in the blood vessel for future blood withdrawal or fluid infusion.

The catheter assembly often includes a luer adapter and a vent plug disposed within the luer adapter. The vent plug provides protection to the luer adapter and catheter assembly, preventing objects from entering the luer adapter and maintaining a sterile environment within the catheter assembly. However, the vent plug often falls off the luer adapter during shipping or transport of the catheter assembly, which results in loss of product integrity and rejection of the catheter assembly for use due to possible contamination.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to a catheter system that includes a locking cap, as well as related devices and methods. In some embodiments, a catheter system may include a catheter and a female luer adapter. In some embodiments, the female luer adapter may include a proximal end and a distal end, and the proximal end of the female luer adapter may include a first protrusion and a second protrusion. In some embodiments, the female luer adapter may be in fluid communication with the catheter.

In some embodiments, the catheter system may include a cap, which may include a top. In some embodiments, the cap may include a male luer extending distally from the top and inserted into the proximal end of the female luer adapter. In some embodiments, the cap may include a first arm extending distally from the top. In some embodiments, the first arm may include a first slot. In some embodiments, the cap may include a second arm extending distally from the top. In some embodiments, the second arm may include a second slot. In some embodiments, the first protrusion may be disposed within the first slot and the second protrusion may be disposed within the second slot. In these embodiments, the cap, which may be referred to in the present disclosure as a “locking cap,” may be disposed in a locked position.

In some embodiments, in response to rotation of the cap with respect to the female luer adapter in a first direction, the first protrusion may be configured to press against an end of the first slot and the second protrusion may be configured to press against an end of the second slot to prevent further rotation of the cap in the first direction. In some embodiments, in response to rotation of the cap with respect to the female luer adapter in a second direction opposite the first direction, the first protrusion may be configured to be removed from the first slot and the second protrusion may be configured to be removed from the second slot.

In some embodiments, the first slot may include a longitudinal edge opposite another longitudinal edge. In some embodiments, the longitudinal edge and/or the other longitudinal edge of the first slot may include a first bump. In some embodiments, the first bump may be interfering with the first protrusion to resist rotation of the cap in the second direction. In some embodiments, the longitudinal edge and/or the other longitudinal edge of the first slot may be angled at a same angle as the first protrusion.

In some embodiments, the second slot may include a longitudinal edge opposite another longitudinal edge. In some embodiments, the longitudinal edge and/or the other longitudinal edge of the second slot may include a second bump. In some embodiments, the second bump may be interfering with the second protrusion to resist rotation of the cap in the second direction. In some embodiments, the longitudinal edge and/or the other longitudinal edge of the second slot may be angled at a same angle as the second protrusion.

In some embodiments, the first protrusion may be opposite the second protrusion. In some embodiments, the first slot may be disposed on a same side of the first arm as the second slot is disposed on the second arm. In some embodiments, the proximal end of the female luer adapter may include a threading, and the first protrusion and the second protrusion may be disposed on the threading. In some embodiments, in a distal end view, the first direction may be clockwise, and the second direction may be counterclockwise.

In some embodiments, the cap may include one or more vents disposed on an outer surface of the male luer. In some embodiments, the one or more vents may be sized and configured to allow air but not liquid to pass. In some embodiments, the one or more vents may extend out of the female luer adapter. In some embodiments, a distal end of the first arm may be chamfered, and a distal end of the second arm may be chamfered.

In some embodiments, the first arm may be spaced apart from the second arm by a gap and another gap opposing the gap. In some embodiments, in response to rotation of the cap with respect to the female luer adapter in the second direction opposite the first direction and removal of the first protrusion from the first slot and the second protrusion from the second slot, the first protrusion may be configured to be disposed within the gap and the second protrusion may be configured to be disposed within the other gap, and the cap may be configured to be removed from the female luer adapter.

In some embodiments, the cap may include a collar extending distally from the top. In some embodiments, an inner surface of the collar may include a first undercut and a second undercut. In some embodiments, the first protrusion may be disposed within the first undercut and the second protrusion may be disposed within the second undercut. In these embodiments, the cap may be disposed in a locked position.

In some embodiments, in response to rotation of the cap with respect to the female luer adapter in the first direction, the first protrusion may be configured to press against an end of the first undercut and the second protrusion may be configured to press against an end of the second undercut to prevent further rotation of the cap in the first direction. In some embodiments, in response to rotation of the cap with respect to the female luer adapter in a second direction opposite the first direction, the first protrusion may be configured to be removed from the first undercut and the second protrusion may be configured to be removed from the second undercut, such that the cap may be removed from the female luer adapter.

In some embodiments, the first undercut may include a longitudinal edge. In some embodiments, the longitudinal edge of the first undercut may include a first bump. In some embodiments, the first bump may be interfering with the first protrusion to resist rotation of the cap in the second direction. In some embodiments, the longitudinal edge of the first undercut may be angled at a same angle as the first protrusion.

In some embodiments, the second undercut may include a longitudinal edge. In some embodiments, the longitudinal edge of the second undercut may include a second bump. In some embodiments, the second bump may be interfering with the second protrusion to resist rotation of the cap in the second direction. In some embodiments, the longitudinal edge of the second undercut may be angled at a same angle as the second protrusion. In some embodiments, a distal end of the collar may be chamfered.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is an upper perspective view of an example catheter system, according to some embodiments;

FIG. 2 is a lower perspective view of an example cap, according to some embodiments;

FIG. 3 is an upper perspective view of the cap, according to some embodiments;

FIG. 4A is an upper perspective view of the cap in a locked position on an example female luer adapter, illustrating the cap including a vent cap, according to some embodiments;

FIG. 4B is a cross-sectional view of the cap in the locked position on the female luer adapter, illustrating the cap including the vent cap, according to some embodiments;

FIG. 5 is an upper perspective view of the cap in the locked position on the female luer adapter, illustrating the female luer adapter including a side port, according to some embodiments;

FIG. 6 is a lower perspective view of the cap and the female luer adapter, illustrating the cap in an unlocked position configured for removal of the female luer adapter, according to some embodiments;

FIG. 7 is a distal end view of the cap and the female luer adapter, illustrating the cap in the unlocked position;

FIG. 8 is a distal end view of the cap and the female luer adapter, illustrating the cap in the unlocked position;

FIG. 9 is a bottom view of the cap, illustrating the top including an example collar, according to some embodiments;

FIG. 10 is an upper perspective view of the cap including the collar and in the locked position, according to some embodiments;

FIG. 11 is a lower perspective view of the cap and the female luer adapter in the unlocked position, illustrating the cap including an example first undercut and an example second undercut, according to some embodiments; and

FIG. 12 is a lower perspective view of the cap, illustrating the cap including an example first undercut and an example second undercut, according to some embodiments.

### DESCRIPTION OF EMBODIMENTS

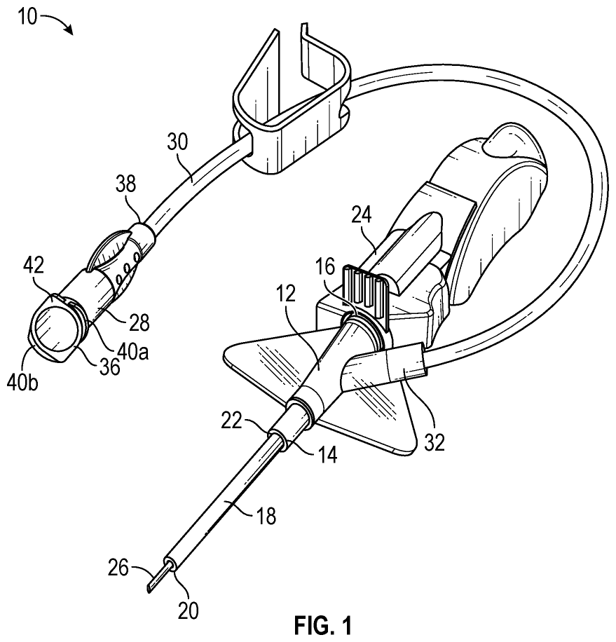

As used in the present disclosure, the term “distal” refers to a portion that is described which is further from a clinician, while the term “proximal” refers to a portion that is being described which is closer to the clinician. Referring now to FIGS. 1, a catheter system 10 is illustrated, according to some embodiments. In some embodiments, the catheter system 10 may include a catheter hub 12, which may include a distal end 14, a proximal end 16, and a catheter hub lumen extending through the distal end 14 of the catheter hub 12 and the proximal end 16 of the catheter hub 12.

In some embodiments, the catheter system 10 may include a catheter 18 extending from the distal end 14 of the catheter hub 12. In some embodiments, the catheter 18 may include a peripheral intravenous catheter (PIVC), a midline catheter, or a peripherally-inserted central catheter. In some embodiments, the catheter 18 may include a catheter tube, which may include a distal end 20, a proximal end 22, and a catheter tube lumen extending through the distal end 20 and the proximal end 22.

In some embodiments, a needle hub 24 may be coupled to the proximal end 16 of the catheter hub 12. In some embodiments, an introducer needle 26 may extend from the needle hub 24. In some embodiments, the needle hub 24 may include a flashback chamber and/or a needle safety device. As illustrated in FIG. 1, in some embodiments, the introducer needle 26 may extend through the catheter hub 12 and the catheter 18 when the catheter system 10 is in an insertion configuration ready for insertion into a patient. In some embodiments, the introducer needle 26 may include a sharp distal tip configured to pierce skin and a vein of the patient. In some embodiments, after the introducer needle 26 is used to place the catheter 18 within the vein, the catheter 18 may be advanced distally further into the vein and/or the introducer needle 26 may then be removed from the catheter 18 and the catheter hub 12.

In some embodiments, the catheter system 10 may include any suitable catheter system. In some embodiments, the catheter system 10 may be similar to the BD SAF-T-INTIMA™ Closed IV Catheter System or the BD NEXIVA™ Closed IV Catheter System or another suitable catheter system in terms of one or more components and/or operation. In some embodiments, the catheter system 10 may include a female luer adapter 28, which may be coupled to or integrated with an extension tube 30 extending from a side port 32 of the catheter hub 12 disposed between the distal end 14 and the proximal end 16.

In some embodiments, the female luer adapter 28 may include a proximal end 36 and a distal end 38. In some embodiments, the proximal end 36 of the female luer adapter 28 may include a female luer. In some embodiments, the proximal end 36 of the female luer adapter 28 may include a first protrusion 40a and a second protrusion 40b. In some embodiments, the proximal end 36 of the female luer adapter 28 may include a threading 42, and the first protrusion 40a and the second protrusion 40b may be disposed on the threading 42.

In some embodiments, the female luer adapter 28 may include a lumen extending therethrough and may be in fluid communication with the catheter 18. In some embodiments, the female luer adapter 28 may be configured to couple to a blood collection device to facilitate collection of blood through the catheter 18 and/or may be configured to couple to an infusion device to deliver fluid to the patient through the catheter 18.

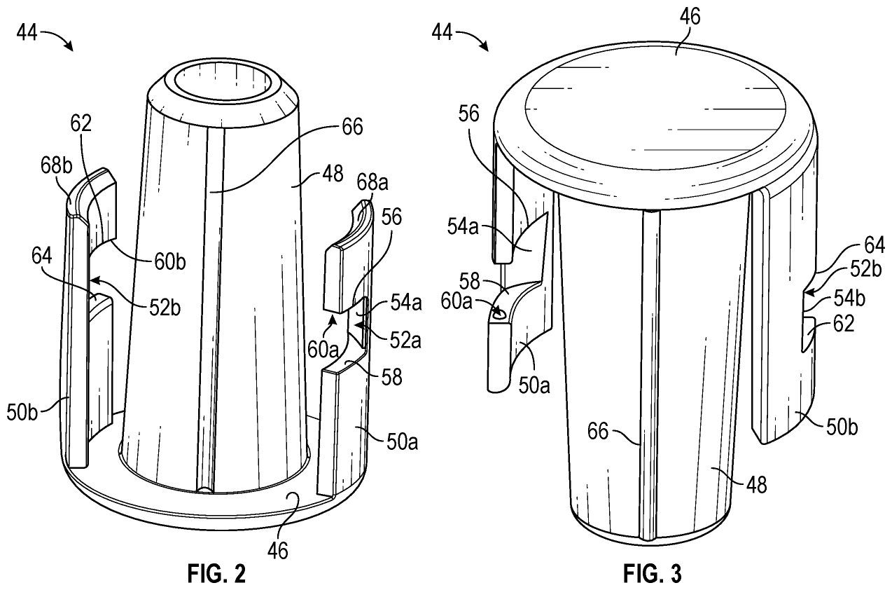

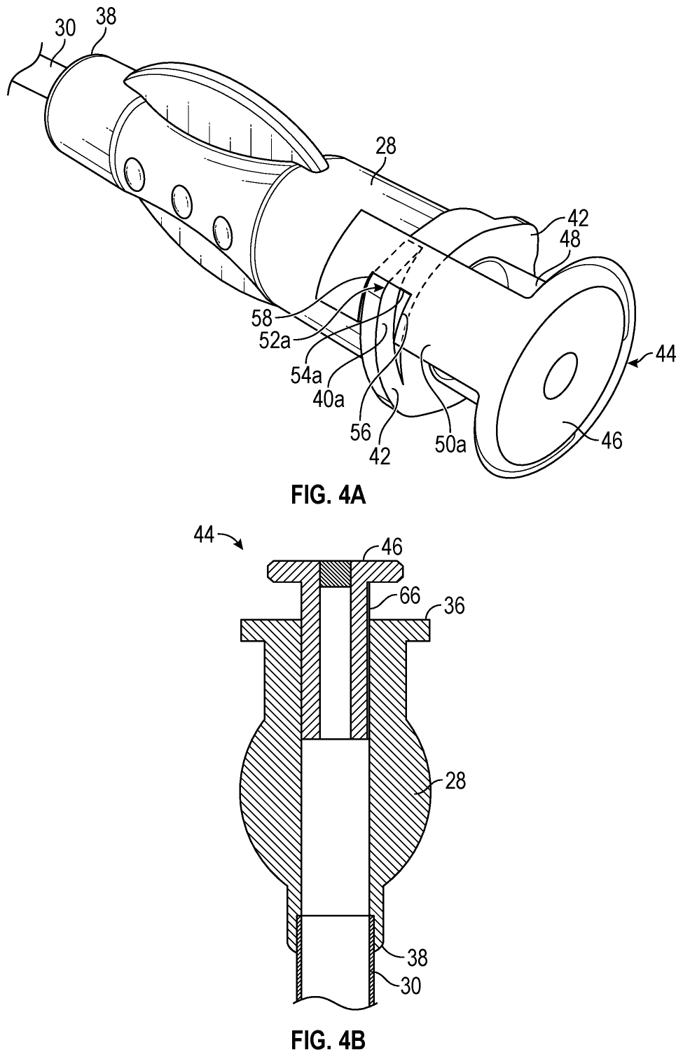

Referring now to FIGS. 2-5, in some embodiments, the catheter system 10 may include a cap 44, which may include a top 46. In some embodiments, the cap 44 may include a male luer 48 extending distally from the top 46 and inserted into the proximal end 36 of the female luer adapter 34. In some embodiments, the cap 44 may include a first arm 50a, which may extend distally from the top 46. In some embodiments, the first arm 50a may include a first slot 52a. In some embodiments, the cap 44 may include a second arm 50b, which may extend distally from the top 46. In some embodiments, the second arm 50b may include a second slot 52b. In some embodiments, the first protrusion 40a may be disposed within the first slot 52a and the second protrusion 40b may be disposed within the second slot 52b. In these embodiments, the cap 44, which may be referred to in the present disclosure as a “locking cap,” may be disposed in a locked position, as illustrated, for example, in FIGS. 4A and 4B. In some embodiments, the cap 44 may be a single piece and monolithically formed as a single unit.

In some embodiments, in response to rotation of the cap 44 with respect to the female luer adapter 28 in a first direction, the first protrusion 40a may be configured to press against an end 54a of the first slot 52a and the second protrusion 40b may be configured to press against an end 54b of the second slot 52b to prevent further rotation of the cap 44 in the first direction. In some embodiments, the first slot 52a may include a longitudinal edge 56 opposite another longitudinal edge 58. In some embodiments, the longitudinal edge 56 and/or the other longitudinal edge 58 of the first slot 52a may include a first bump 60a. In some embodiments, the first bump 60a may be interfering with the first protrusion 40a to resist rotation of the cap 44 in a second direction opposite the first direction. In some embodiments, the longitudinal edge 56 and/or the other longitudinal edge 58 of the first slot 52a may be angled at a same angle as the first protrusion 40a, which may facilitate twisting of the cap 44 between the locked position and an unlocked position.

In some embodiments, the second slot 52b may include a longitudinal edge 62 opposite another longitudinal edge 64. In some embodiments, the longitudinal edge 62 and/or the other longitudinal edge 64 of the second slot 52b may include a second bump 60b. In some embodiments, the second bump 60b may be interfering with the second protrusion 40b to resist rotation of the cap 44 in the second direction. In some embodiments, the longitudinal edge 62 and/or the other longitudinal edge 64 of the second slot 52b may be angled at a same angle as the second protrusion 40b, which may facilitate twisting of the cap 44 between the locked position and the unlocked position. In some embodiments, the first bump 60a and the second bump 60b may enhance locking of the cap 44 with respect to the female luer adapter 34.

In some embodiments, the first protrusion 40a may be opposite the second protrusion 40b. In some embodiments, the first slot 52a may be disposed on a same side of the first arm 50a as the second slot 52b is disposed on the second arm 50b, which may facilitate movement of the cap 44 between the locked position and the unlocked position. In further detail, in some embodiments, the first slot 52a may extend from a right edge of the first arm 50a, and the second slot 52b may extend from a right edge of the second arm 50b.

In some embodiments, the cap 44 may include one or more vents 66 disposed on an outer surface of the male luer 48. In some embodiments, the one or more vents 66 may be sized and configured to allow air but not liquid to pass. Thus, the cap 44 disposed within the female luer adapter 28 may create a liquid seal. In some embodiments, the one or more vents 66 may extend out of the female luer adapter 28. In some embodiments, the one or more vents 66 may facilitate blood flashback in response to insertion of the catheter 18 into the vein of the patient.

In some embodiments, a distal end 68a of the first arm 50a may be chamfered, and a distal end 68b of the second arm 50b may be chamfered. In some embodiments, the first arm 50a chamfered and the second arm 50b chamfered may facilitate placement of the cap 44 over the female luer adapter 34, such as the first protrusion 40a and the second protrusion 40b, during assembly and/or removal of the cap 44.

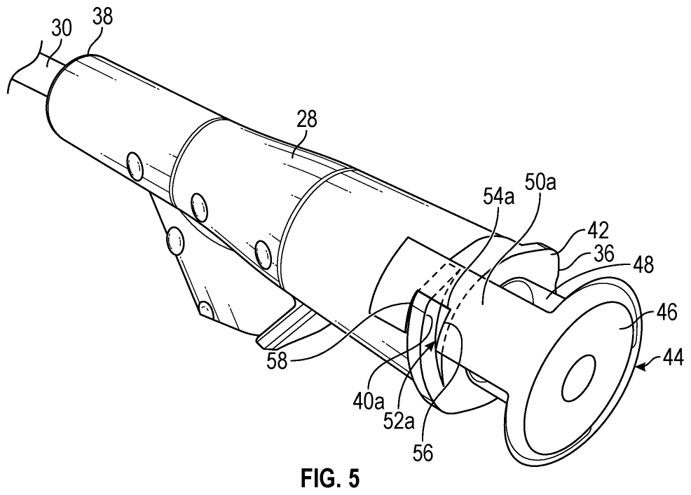

In some embodiments, the cap 44 may include a vent cap, as illustrated, for example, in FIGS. 4A-5. In some embodiments, the vent cap may include a porous material or membrane that may be configured to allow air but not fluid to exit the female luer adapter 34. In other embodiments, as illustrated, for example, in FIGS. 2-3, the cap 44 may include an end cap, and the top 46 may be solid.

In some embodiments, the female luer adapter 34 may include a side port, as illustrated, for example, in FIG. 5. In other embodiments, the female luer adapter 34 may not include the side port, as illustrated, for example, in FIG. 4. In some embodiments, in response to rotation of the cap 44 with respect to the female luer adapter 34 in the second direction opposite the first direction, the first protrusion 40a may be removed from the first slot 52a and the second protrusion 40b may be removed from the second slot 52b such that the cap 44 is in the unlocked position, which may facilitate removal of the cap 44.

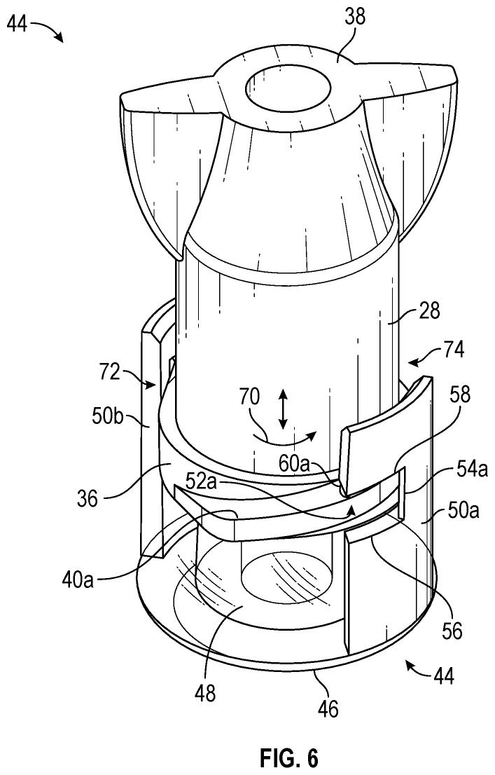

Referring now to FIG. 6, in some embodiments, the first arm 50a may be spaced apart from the second arm 50b by a gap 72 and another gap 74 opposing the gap 72. In some embodiments, in response to rotation of the cap 44 with respect to the female luer adapter 34 in the second direction opposite the first direction and removal of the first protrusion 40a from the first slot 52a and the second protrusion 40b from the second slot 52b, the first protrusion 40a may be disposed within the gap 72 and the second protrusion 40b may be disposed within the other gap 74. In some embodiments, when the first protrusion 40a is disposed within the gap 72 and the second protrusion 40b is disposed within the other gap 74, as illustrated, for example, in FIG. 6, the cap 44 may be unlocked and configured to be removed from the female luer adapter 28.

Referring now to FIG. 7, in some embodiments, in response to rotation of the cap 44 with respect to the female luer adapter 34 in a second direction 70 opposite the first direction 71, the first protrusion 40a may be removed from the first slot and the second protrusion 40b may be removed from the second slot. In some embodiments, the first protrusion 40a removed from the first slot 52a and the second protrusion 40b removed from the second slot 52b may unlock the cap 44, which may be then be removed from the female luer adapter 28. Thus, in some embodiments, the cap 44 may be coupled to the female luer adapter 28 through a “push then rotate” movement by the clinician and removed from the female luer adapter 28 through a “rotate then pull” movement by the clinician.

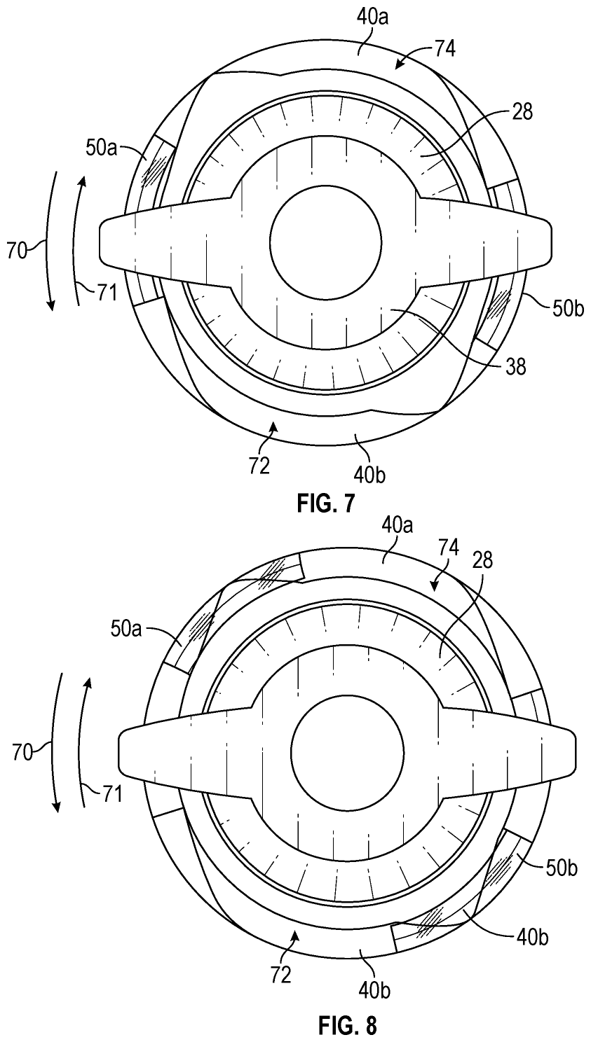

Referring now to FIG. 8, in some embodiments, the first protrusion 40a may be disposed within the first slot and the second protrusion 40b may be disposed within the second slot. In these embodiments, the cap 44 may be disposed in a locked position, with the first slot and the second slot preventing removal of the cap 44. In some embodiments, in a distal end view, as illustrated, for example, in FIGS. 7-8, the first direction 71 may be clockwise, and the second direction 70 may be counterclockwise, which may facilitate use with the threading 42, which may be right-handed.

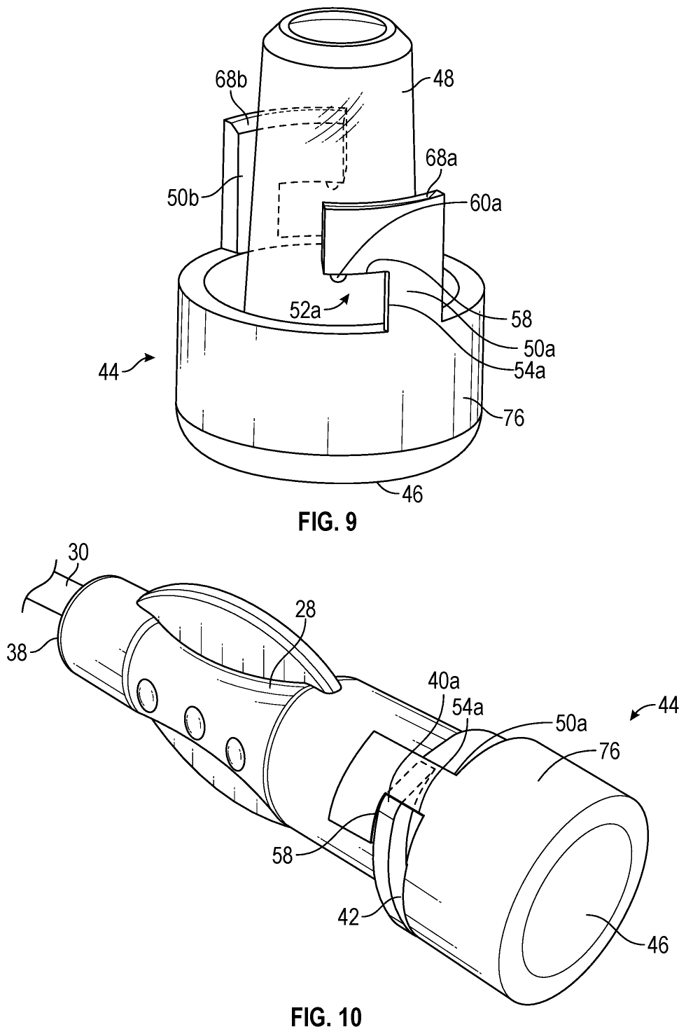

Referring now to FIGS. 9-10, in some embodiments, the cap 44 may include a collar 76 extending distally from the top 46. In these embodiments, the first arm 50a and the second arm 50b may extend distally from the collar 76, and the first slot 52a and the second slot 52b may be disposed between the collar 76 and the first arm 50a and the second arm 50b, respectively. In some embodiments, the collar 76 may facilitate a closed system.

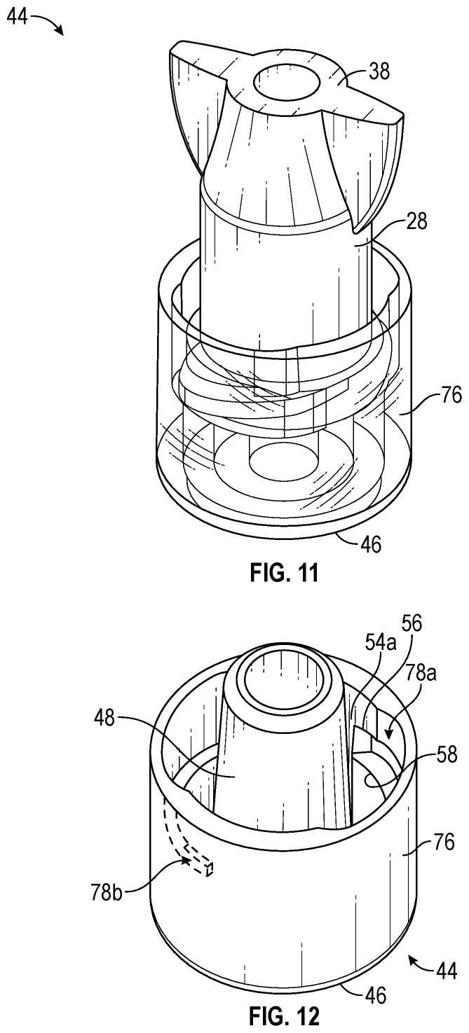

Referring now to FIGS. 11-12, in some embodiments, the cap may include the collar 76 extending distally from the top 46. In some embodiments, an inner surface of the collar 76 may include a first undercut 78a and a second undercut 78b, which may function similar to the first slot 52a and the second slot 52b of FIGS. 2-10. In some embodiments, the second undercut 78b may include a similar shape as the first undercut 78a. In some embodiments, the first protrusion 40a may be disposed within the first undercut 78a and the second protrusion 40b (see, for example, FIGS. 1-10) may be disposed within the second undercut 78b. In these embodiments, the cap 44 may be disposed in the locked position.

In some embodiments, in response to rotation of the cap 44 with respect to the female luer adapter 28 in the first direction, the first protrusion 40a may be configured to press against an end 54a of the first undercut 78a and the second protrusion 40b may be configured to press against an end of the second undercut to prevent further rotation of the cap 44 in the first direction. In some embodiments, in response to rotation of the cap 44 with respect to the female luer adapter 28 in a second direction opposite the first direction, the first protrusion 40a may be configured to be removed from the first undercut 78a and the second protrusion 40b may be configured to be removed from the second undercut 78b, such that the cap 44 may be unlocked and removed from the female luer adapter 28.

In some embodiments, the first undercut 78a may include a longitudinal edge 56. In some embodiments, the longitudinal edge 56 of the first undercut 78a may include a first bump, such as the first bump 60a illustrated, for example, in FIGS. 2-3. In some embodiments, the first bump may be interfering with the first protrusion 40a to resist rotation of the cap 44 in the second direction. In some embodiments, the longitudinal edge 56 and/or another longitudinal edge 58 of the first undercut 78a may be angled at a same angle as the first protrusion 40a, which may facilitate twisting of the cap 44 between the locked position and the unlocked position.

In some embodiments, the second undercut 78b may include a longitudinal edge and another longitudinal edge similar to the first undercut 78a. In some embodiments, the longitudinal edge of the second undercut 78b may include a second bump such as the second bump 60b illustrated, for example, in FIG. 2. In some embodiments, the second bump may be interfering with the second protrusion 40b to resist rotation of the cap 44 in the second direction. In some embodiments, the longitudinal edge and the other longitudinal edge of the second undercut 78b may be angled at a same angle as the second protrusion, which may facilitate twisting of the cap 44 between the locked position and the unlocked position. In some embodiments, a distal end of the collar 76 may be chamfered.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A catheter system, comprising:
a catheter;
a female luer adapter comprising a proximal end and a distal end, wherein the proximal end comprises a first protrusion and a second protrusion, wherein the female luer adapter is in fluid communication with the catheter;
a cap, comprising:
a top;
a male luer extending distally from the top and inserted into the proximal end of the female luer adapter;
a first arm extending distally from the top, wherein the first arm comprises a first slot;
a second arm extending distally from the top, wherein the second arm comprises a second slot, wherein the first protrusion is disposed within the first slot and the second protrusion is disposed within the second slot,
wherein in response to rotation of the cap with respect to the female luer adapter in a first direction, the first protrusion is configured to press against an end of the first slot and the second protrusion is configured to press against an end of the second slot to prevent further rotation of the cap in the first direction, wherein in response to rotation of the cap with respect to the female luer adapter in a second direction opposite the first direction, the first protrusion is configured to be removed from the first slot and the second protrusion is configured to be removed from the second slot.

2. The catheter system of claim 1, wherein the first slot comprises a longitudinal edge opposite another longitudinal edge, wherein the longitudinal edge or the other longitudinal edge of the first slot comprises a first bump, wherein the first bump is interfering with the first protrusion to resist rotation of the cap in the second direction.

3. The catheter system of claim 2, wherein the second slot comprises a longitudinal edge opposite another longitudinal edge, wherein the longitudinal edge or the other longitudinal edge of the second slot comprises a second bump, wherein the second bump is interfering with the second protrusion to resist rotation of the cap in the second direction.

4. The catheter system of claim 1, wherein the first slot comprises a longitudinal edge opposite another longitudinal edge, wherein the longitudinal edge and the other longitudinal edge of the first slot are angled at a same angle as the first protrusion.

5. The catheter system of claim 4, wherein the second slot comprises a longitudinal edge opposite another longitudinal edge, wherein the longitudinal edge and the other longitudinal edge of the second slot are angled at a same angle as the second protrusion.

6. The catheter system of claim 1, wherein the first protrusion is opposite the second protrusion.

7. The catheter system of claim 1, wherein the first slot is disposed on a same side of the first arm as the second slot is disposed on the second arm.

8. The catheter system of claim 1, wherein the proximal end of the female luer adapter comprises a threading, wherein the first protrusion and the second protrusion are disposed on the threading.

9. The catheter system of claim 1, wherein in a distal end view, the first direction is clockwise, wherein the second direction is counterclockwise.

10. The catheter system of claim 1, wherein the cap further comprises a plurality of vents disposed on an outer surface of the male luer, wherein the vents are sized to allow air but not liquid to pass, wherein the plurality of vents extend out of the female luer adapter.

11. The catheter system of claim 1, wherein a distal end of the first arm is chamfered, wherein a distal end of the second arm is chamfered.

12. The catheter system of claim 1, wherein the first arm is spaced apart from the second arm by a gap and another gap opposing the gap, wherein in response to rotation of the cap with respect to the female luer adapter in the second direction opposite the first direction and removal of the first protrusion from the first slot and the second protrusion from the second slot, the first protrusion is configured to be disposed within the gap and the second protrusion is configured to be disposed within the other gap, and the cap is configured to be removed from the female luer adapter.

13. A catheter system, comprising:
a catheter;
a female luer adapter comprising a proximal end and a distal end, wherein the proximal end comprises a first protrusion and a second protrusion, wherein the female luer adapter is in fluid communication with the catheter;
a cap, comprising:
a top;
a male luer extending distally from the top and inserted into the proximal end of the female luer adapter;
a collar extending distally from the top, wherein an inner surface of the collar comprises a first undercut and a second undercut, wherein the first protrusion is disposed within the first undercut and the second protrusion is disposed within the second undercut,
wherein in response to rotation of the cap with respect to the female luer adapter in a first direction, the first protrusion is configured to press against an end of the first undercut and the second protrusion is configured to press against an end of the second undercut to prevent further rotation of the cap in the first direction, wherein in response to rotation of the cap with respect to the female luer adapter in a second direction opposite the first direction, the first protrusion is configured to be removed from the first undercut and the second protrusion is configured to be removed from the second undercut, and the cap is configured to be removed from the female luer adapter.

14. The catheter system of claim 13, wherein the first undercut comprises a longitudinal edge, wherein the longitudinal edge of the first undercut comprises a first bump, wherein the first bump is interfering with the first protrusion to resist rotation of the cap in the second direction.

15. The catheter system of claim 14, wherein the second undercut comprises a longitudinal edge, wherein the longitudinal edge of the second undercut comprises a second bump, wherein the second bump is interfering with the second protrusion to resist rotation of the cap in the second direction.

16. The catheter system of claim 13, wherein the first undercut comprises a longitudinal edge, wherein the longitudinal edge of the first undercut is angled at a same angle as the first protrusion.

17. The catheter system of claim 16, wherein the second undercut comprises a longitudinal edge, wherein the longitudinal edge of the second undercut is angled at a same angle as the second protrusion.

18. The catheter system of claim 13, wherein in a distal end view, the first direction is clockwise, wherein the second direction is counterclockwise.

19. The catheter system of claim 13, wherein the cap further comprises a plurality of vents disposed on an outer surface of the male luer, wherein the vents are sized to allow air but not liquid to pass, wherein the plurality of vents extend out of the female luer adapter.

20. The catheter system of claim 13, wherein a distal end of the collar is chamfered.
